(19) United States
(12) Patent Application Publication
(10) Pub. No.: US 2005/0147258 A1
(43) Pub. Date: Jul. 7, 2005

(54) Method for adjusting adaptation control of adaptive interference canceller

(76) Inventors: Ville Myllyla, Tampere (FI); Matti Kajala, Tampere (FI); Matti Hamalainen, Lempaala (FI)

Correspondence Address: WARE FRESSOLA VAN DER SLUYS &;ADOLPHSON, LLP, BRADFORD GREEN BUILDING 5, 755 MAIN STREET, P O BOX 224, MONROE, CT, 06468, US

(21) Appl. No.: 10/746,843

(22) Filed: Dec. 24, 2003

(51) Int. Cl. A61F 11/06; G10K 11/16; H03B 29/00; G06F 17/10

(52) U.S. Cl. 381/71.11; 708/322; 381/94.2

(57) ABSTRACT

The present invention describes a method for temporal adjustment of adaptation control of an adaptive interference canceller (AIC) based on spatially weighted beamforming pre-processing. Most importantly, the present invention enhances the spatial blocking performance, while generating noise references for the AIC by a beamformer, by introducing dynamic adjustment to the AIC filter adaptation control. As a result, adaptation is effectively realized in two dimensions—spatial and temporal. The criterion for adjusting the AIC filter adaptation control is applied continuously following generation of the noise references. Essential in the invention is the comparison of the short-time powers or levels of the noise reference signals and desired signal beams and allowing the adaptation of the AIC filter under consideration only when the noise reference signal power is large enough in comparison with the desired signal power.

### CROSS-REFERENCE TO RELATED APPLICATION

This application discloses subject matter which is also disclosed and which may be claimed in co-pending, co-owned applications (Att. Doc. No 944-003.196 and 44-003.197) filed on even date herewith.

### FIELD OF THE INVENTION

This invention generally relates to acoustic signal processing and more specifically to preventing adaptive interference canceller from canceling the desired speech signal by dynamic adjustment of adaptation control based on beamforming pre-processing.

### BACKGROUND OF THE ART

1. Field of Technology and Background

A beam, referred to in the present invention, is a processed output target signal of multiple receivers. A beamformer is a spatial filter that processes multiple input signals (spatial samples of a wave field) and provides a single output picking up the desired signal while filtering out the signals coming from other directions. The term adaptive beamformer refers to a well-known generalized sidelobe canceller (GSC), which is a combination of a beamformer providing the desired signal output and an adaptive interference canceller (AIC) part that produces noise estimates that are then subtracted from the desired signal output, further reducing any ambient noise left there on the desired signal path. Desired signal is, e.g. a speech signal coming from the direction of the source and noise signals are all other signals present in the environment including reverberated components of the desired signal. Reverberation occurs when a signal (acoustical pressure wave or electromagnetic radiation) hits an obstacle and changes its direction, possibly reflecting back to the system from another direction.

2. Problem Formulation

In conventional GSC systems the desired signal is prevented from the AIC inputs by means of a so-called blocking matrix as described, e.g., by Claesson and Nordholm, “A Spatial Filtering Approach to Robust Adaptive Beaming”, IEEE Trans. on Antennas and Propagation, Vol. 40, No. 9, September 1992. Otherwise, the desired signal will also be cancelled. However, due to imperfect realizations of such systems the desired signal leaks to the AIC filter inputs that cause the desired signal deterioration in the system output.

3. Prior Art

In conventional GSCs, it could be possible to try preventing a desired signal cancellation by restricting the performance of the adaptive filters (e.g. leaky LMS, least-mean-square) and/or widening the spatial angle used for blocking. Also, it is possible to add temporal constraints to the blocking matrix to try to enhance the desired signal blocking.

Additionally, temporal adaptation control for AIC filters can be implemented with the help of the voice activity detector (VAD). The detector is placed after the beam looking to the desired direction. When speech from the desired direction is detected the adaptation of the AIC filters is prevented.

Prior-art solutions are sub-optimal in a sense that they (e.g., leaky LMS adaptive filters) may not provide as good interference cancellation as would be possible without restricting the performance of the adaptive filter.

Temporal constraints in the blocking matrix may enhance the blocking performance, but it is unclear how to generate and adapt the constraints in the sensible manner. Furthermore, they add complexity to the blocking operation, which is already rather complex, especially, in case of beam steering (changing the look direction of the beamformer).

Also, the blocking matrix is conventionally formed as a filter that is calculated as a complement to the beamforming filter and, therefore, changing the look (target) direction of the beamformer requires typically a rather exhaustive recalculation of the complementary filter when the desired signal source moves around. On the other hand, complementary filters could be stored in a memory, which requires that filter coefficients are stored separately for each look direction. In that case, the actual look (target) direction of the beamformer is restricted to the look (target) directions obtained from the pre-calculated filters in the memory. One more alternative is to use pre-steering of the array signals towards the desired signal source (the desired signal is in-phase in all channels). However, pre-steering requires either analog delays or digital fractional delay filters, which, in turn, are rather long and therefore complex to implement.

VAD (voice activity detector) based temporal constraining relies on the VAD performance, which is often unreliable. Furthermore, the VADs are rather complex to implement, especially, in terms of robustness and reliability.

### SUMMARY OF THE INVENTION

The objective of the present invention is to provide a novel method for dynamic adjustment of adaptation control of an adaptive interference canceller based on spatially weighted beamforming pre-processing.

According to a first aspect of the present invention, a method for dynamic adjustment of adaptation control of an adaptive interference canceller based on spatially weighted beamforming pre-processing, comprises the steps of: generating a target signal and N noise reference signals by a beamformer and providing said target signal and said N noise reference signals to the adaptive interference canceller, wherein N is a finite integer of at least a value of one; calculating by the adaptive interference canceller N noise-to-target estimate signals and comparing, according to a predetermined criterion, each of the N noise-to-target estimate signals with a corresponding one of N adjustment thresholds, respectively, and optionally with at least one further adjustment threshold, wherein said at least one further adjustment threshold is selected individually for each of the N noise-to-target estimate signals; providing, based on said predetermined criterion, each of N adjustment signals to a corresponding one of N adaptive filter blocks of the adaptive interference canceller, respectively; generating each of N noise cancellation adaptive signals by the corresponding one of the N adaptive filter blocks based on a corresponding one of the N adjustment signals; and generating an output target signal by subtracting all N noise cancellation adaptive signals from the target signal.

In further accord with the first aspect of the invention, the beamformer may be a polynomial beamformer.

Still further according to the first aspect of the invention, the target signal is generated by a target post-filter of the beamformer in response to each of T+1 intermediate signals and to a target control signal, and each of the N noise reference signals is generated by each of the N noise post-filters of the beamformer in response to the T+1 intermediate signals and to a corresponding one of the N noise control signals provided to a corresponding one of the N noise post-filters, respectively, said T+1 intermediate signals are generated by T+1 pre-filters of the beamformer, each of said T+1 pre-filters is responsive to M microphone signals or to M digital microphone signals and said target control signal and said noise control signals are generated by a beam shape control block of the beamformer, wherein M is a finite integer of at least a value of two and T is a finite integer of at least a value of one. Further, the M microphone signals may be generated by a microphone array containing M microphones, responsive to an acoustic signal. Still further, the M digital microphone signals may be generated by an A/D converter from the M microphone signals provided by the microphone array.

Further still according to the first aspect of the invention, the target signal and a corresponding one of said N noise reference signals are provided to each of N noise-to-target estimators of the adaptive interference canceller, respectively, and each of N noise-to-target estimate signals is calculated by a corresponding one of the N noise-to-target estimators as a ratio of the corresponding one of the N noise reference signals, respectively, and the target signal.

In further accordance with the first aspect of the invention, each of the N adjustment signals is a true/false control signal and each of the N noise-to-target estimate signals is only compared, according to a predetermined criterion, with the corresponding one of the N adjustment thresholds, respectively. Further, all of the N adjustment thresholds may be equal to each other and to a common adjustment threshold R₀, said common adjustment threshold may be in the range 0.5≦R₀≦2.0. Still further, each of the N true/false control signals may be determined by comparing a corresponding one of the N noise-to-target estimate signals with the corresponding one of the N adjustment thresholds, respectively, such that if any of said noise-to-target estimate signals is larger than the corresponding one of the N adjustment thresholds, the corresponding true control signal may be provided to a corresponding one of the N adaptive filter blocks, respectively, but if any of said N noise-to-target estimate signals is smaller than the corresponding one of the N corresponding adjustment thresholds, then the false control signal may be provided to the corresponding one of the N adaptive filter blocks, respectively. Yet further, each of the N true/false control signals may be used for adjusting an adaptation rate of the corresponding one of the N adaptive filter blocks of the adaptive interference canceller, respectively. Still further, the N true/false control signals may be provided to the N adaptive filter blocks for enabling or disabling the adaptation procedure of the adaptation coefficients to allow generation of new adaptation coefficients in case of the true control signals or freezing of said adaptation coefficients in case of the false control signal by each of the N adaptive filter blocks.

Yet further still according to the first aspect of the invention, each of the N true/false control signals is determined by comparing a corresponding one of the N noise-to-target estimate signals with the corresponding one of the N adjustment thresholds, respectfully, such that if any of said noise-to-target estimate signals is larger than the corresponding one of the N adjustment thresholds, the corresponding true control signal is provided to a corresponding one of the N adaptive filter blocks, respectively, but if any of said N noise-to-target estimate signals is smaller than the corresponding one of the N corresponding adjustment thresholds, then the false control signal is provided to the corresponding one of the N adaptive filter blocks, respectively. Still further, the N adaptive filters may be finite impulse response (FIR) filters.

According still further to the first aspect of the invention, the subtraction of the N noise cancellation adaptive signals from the target signal for generating the output target signal may be performed by N adders sequentially by generating N−1 corresponding intermediate output target signals. Alternatively, said subtraction may be performed by a combined adder.

According further to the first aspect of the invention, the output target signal is provided to each of the N adaptive filter blocks for continuing an adaptation process and for generating a further value of the output target signal.

According still further to the first aspect of the invention, the at least one of the N adjustment thresholds or of the at least one further adjustment threshold for each of the N noise-to-target estimate signals may be variable as a function of time according to a further predetermined criteria. Alternatively, all of said N adjustment thresholds and the at least one further adjustment threshold for each of the N noise-to-target estimate signals may be variable as a function of time according to a further predetermined criteria. Further alternatively, said at least one further adjustment threshold for each of the N noise-to-target estimate signals may be equal to a further common adjustment threshold.

According further still to the first aspect of the invention, each of the N adjustment signals may be used for adjusting an adaptation rate of the corresponding one of the corresponding N adaptive filter blocks of the adaptive interference canceller, respectively.

Yet still further according to the first aspect of the invention, N may be equal to one and/or an adaptive interference cancellation may be performed in a frequency domain, or in a time domain or in both the frequency and the time domain.

According to a second aspect of the invention, a generalized sidelobe canceling system comprises: a beamformer, for providing a target signal and N noise reference signals, wherein N is a finite integer of at least a value of one; and an adaptive interference canceller, responsive to the target signal, to N noise reference signals and to an output target signal, for adjusting adaptation control of the output target signal based on calculating N noise-to-target estimate signals and comparing, according to a predetermined criterion, each of said N noise-to-target estimate signals with a corresponding one of N adjustment threshold, respectively and optionally with at least one further adjustment threshold, wherein said at least one further adjustment threshold is selected individually for each of the noise-to-target estimate signals.

According further to the second aspect of the invention, the beamformer may be a polynomial beamformer.

Further according to the second aspect of the invention, the generalized sidelobe canceling system further comprises: a microphone array containing M microphones, responsive to an acoustic signal, for providing M microphone signals, wherein M is a finite integer of at least a value of two; an A/D converter, responsive to the M microphone signals, for providing M digital microphone signals; and a speaker and noise tracking block, responsive to the T+1 intermediate signals, for providing a direction of arrival signal and N noise direction signals, wherein T is a finite integer of at least a value of one. Further, the beamformer may be responsive to the M microphone signals or to the M digital microphone signals and optionally responsive to the direction of arrival signal and to the N noise direction signals, for providing T+1 intermediate signals, a target control signal and N noise control signals. Still further, the beamformer may comprise: T+1 pre-filters, responsive to the M digital microphone signal, for providing the T+1 intermediate signals; N target post-filters, responsive to the T+1 intermediate signals and to the target control signal, for providing the target signal; N noise post-filters, each responsive to the T+1 intermediate signals and to a corresponding one of the N noise control signals, each for providing a corresponding one of the N noise reference signals; and a beam shape control block, optionally responsive to the direction of arrival signal and to the N noise direction signals, for providing the target control signal and the N noise control signals.

Still further according to the second aspect of the invention, the adaptive interference canceller comprises: N adaptive filter blocks, each responsive to the output target signal, to a corresponding one of N adjustment signals and to a corresponding one of the N noise reference signals, respectively, each for providing one of N noise cancellation adaptive signals by a corresponding one of the N adaptive filter blocks; N consecutive adders, each responsive to the target signal and to the corresponding one of the N noise cancellation adaptive signals, respectively, each for providing one of N−1 corresponding intermediate signals or the output target signal by a corresponding one of the N adders, respectively; and N adaptation control adjustment blocks, each responsive to the target signal and to the corresponding one of the N noise reference signals, respectively, each for providing one of the N corresponding adjustment signals by a corresponding one of the N adaptation control adjustment blocks. Further, each of the adaptive filter blocks may comprise: an adaptive filter, responsive to the corresponding one of the N noise reference signals and to a corresponding one of N coefficient signals, respectively, for providing a corresponding one of the N noise cancellation adaptive signals by each of the adaptive filters, respectively; and a coefficient adaptation block, responsive to the corresponding one of the N noise reference signals responsive to the output target signal, for providing one of the N coefficient signals by a corresponding one of the coefficient adaptation blocks, respectively. Yet still further, each of the N adaptation control adjustment blocks may comprise: a noise-to-target estimator, responsive to the target signal and to the corresponding one of the N noise reference signals, respectively, for providing a corresponding one of the N noise-to-target estimate signals, respectively; and an adjustment controller, responsive to the corresponding one of the N noise-to-target estimate signals, for providing a corresponding one of the N adjustment signals, respectively.

According further still to the second aspect of the invention, all N of the adjustment thresholds may be equal to each other and to a common adjustment threshold R₀, said common adjustment threshold may be in the range 0.5≦R₀≦2.0.

Yet still further according to the second aspect of the invention, N may be equal to one and/or the generalized sidelobe canceling system may be implemented in a frequency domain, or in a time domain or in both the frequency and the time domain.

According to a third aspect of the invention, an adaptive interference canceller for generating an output target signal with dynamic adjustment of adaptation control comprises: N adaptive filter blocks, each responsive to the output target signal, and to a corresponding one of N adjustment signals and to a corresponding one of N noise reference signals, each for providing one of N noise cancellation adaptive signals by a corresponding one of the N adaptive filter blocks; and N adaptation control adjustment blocks, each responsive to the target signal and to the corresponding one of the N noise reference signals, respectively, each for providing one of the N adjustment signals by a corresponding one of the N adaptation control adjustment blocks. Further, the adaptive interference canceller may further comprise: N consecutive adders, each responsive to the target signal and to a corresponding one of the N noise cancellation adaptive signals, respectively, for providing one of N−1 corresponding intermediate signals or the output target signal by a corresponding one of the N adders, respectively. Still further, each of the N adaptive filter blocks may comprise: an adaptive filter, responsive to a corresponding one of the N noise reference signals and to a corresponding one of N coefficient signals, respectively, each for providing one of the N noise cancellation adaptive signals by a corresponding one of the N adaptive filters, respectively; and a coefficient adaptation block, responsive to the corresponding one of the N noise reference signals and to the output target signal, for providing one of the N coefficient signals by a corresponding one of the coefficient adaptation blocks, respectively. Yet still further, each of the N adaptation control adjustment blocks may comprise: a noise-to-target estimator, responsive to the target signal and to a corresponding one of the N noise reference signals, respectively, for providing a corresponding one of N noise-to-target estimate signals, respectively; and an adjustment controller, responsive to the corresponding one of the N noise-to-target estimate signals, for providing a corresponding one of the N adjustment signals, respectively.

### BRIEF DESCRIPTION OF THE DRAWINGS

For a better understanding of the nature and objects of the present invention, reference is made to the following detailed description taken in conjunction with the following drawings, in which:

FIGS. 1a and 1b together demonstrate a block diagram representing an example of generalized sidelobe canceling with N noise reference signals using dynamic adjustment of adaptation control of an adaptive interference canceller based on spatially weighted beamforming pre-processing: FIG. 1 a shows components of a generalized sidelobe canceling system including a polynomial beamformer, which generates N noise reference signals supporting operation of adaptive interference cancellers shown in FIG. 1b, according to the present invention.

FIGS. 2a, 2b and 2c illustrate different examples of distribution of a target direction and noise reference directions, according to the present invention.

FIG. 3 is a block diagram representing an example of generalized sidelobe canceling with one noise reference signal using dynamic adjustment of adaptation control of an adaptive interference canceller based on spatially weighted beamforming pre-processing, according to the present invention.

FIG. 4 shows a flow chart of generalized sidelobe canceling using dynamic adjustment of adaptation control of an adaptive interference canceller based on spatially weighted beamforming pre-processing, according to the present invention.

### BEST MODE FOR CARRYING OUT THE INVENTION

The present invention provides a method for dynamic adjustment of adaptation control of an adaptive interference canceller (AIC) based on spatially weighted beamforming pre-processing. First of all, the present invention takes an advantage of the polynomial beamformer described in European Patent No. 1184676 “A method and a Device for Parametric Steering of a Microphone Array Beamformer” by M. Kajala and M. Hämäiläinen (corresponding PCT Patent Application publication WO 02/18969), for generating noise references for AIC filters providing spatial blocking of the desired signal while generating noise references for AIC filters using spatially weighted beamforming pre-processing. Most importantly, the present invention enhances the blocking performance furthermore by introducing dynamic temporal constraints to adjust the AIC filter adaptation control. As a result, the blocking is effectively realized in two dimensions—spatial and temporal. The constraints are calculated continuously and applied after generating the noise references. Thus, as a serial and separate process the present invention does not complicate the previous processes. Essential in the invention is the comparison of the short-time powers or other signal levels indicative of a performance of the noise reference signals and desired signal beams and allowing adjustment of the adaptation control of the AIC filter under consideration only when the noise reference signal level is large enough in comparison with the desired signal level. At last but not least, the simple structure of the invention provides robust and reliable performance of the constraints and very efficient implementation assuming that the beam and the noise references are available.

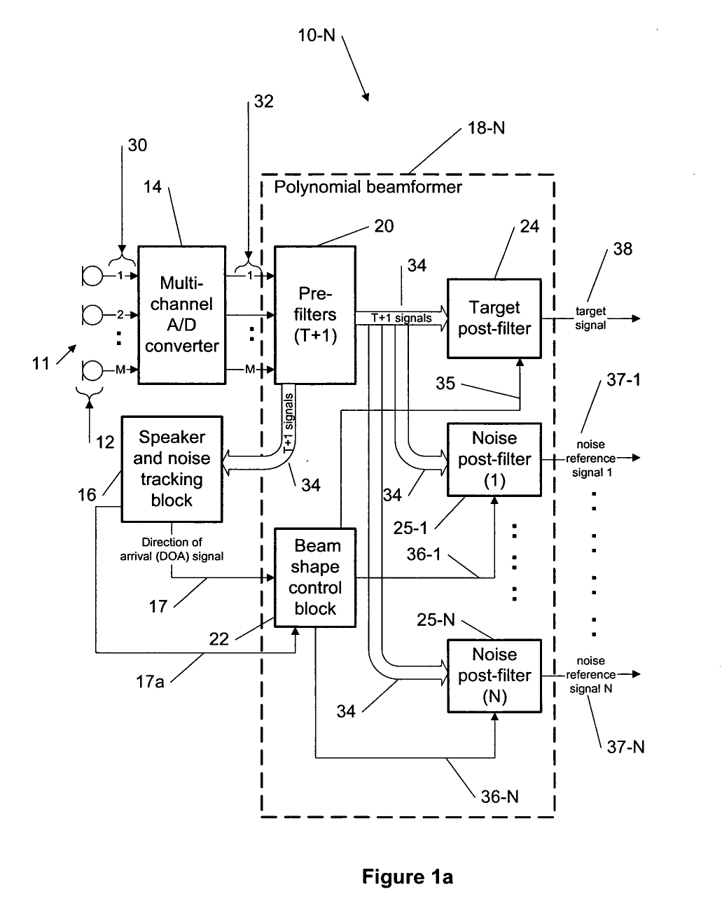

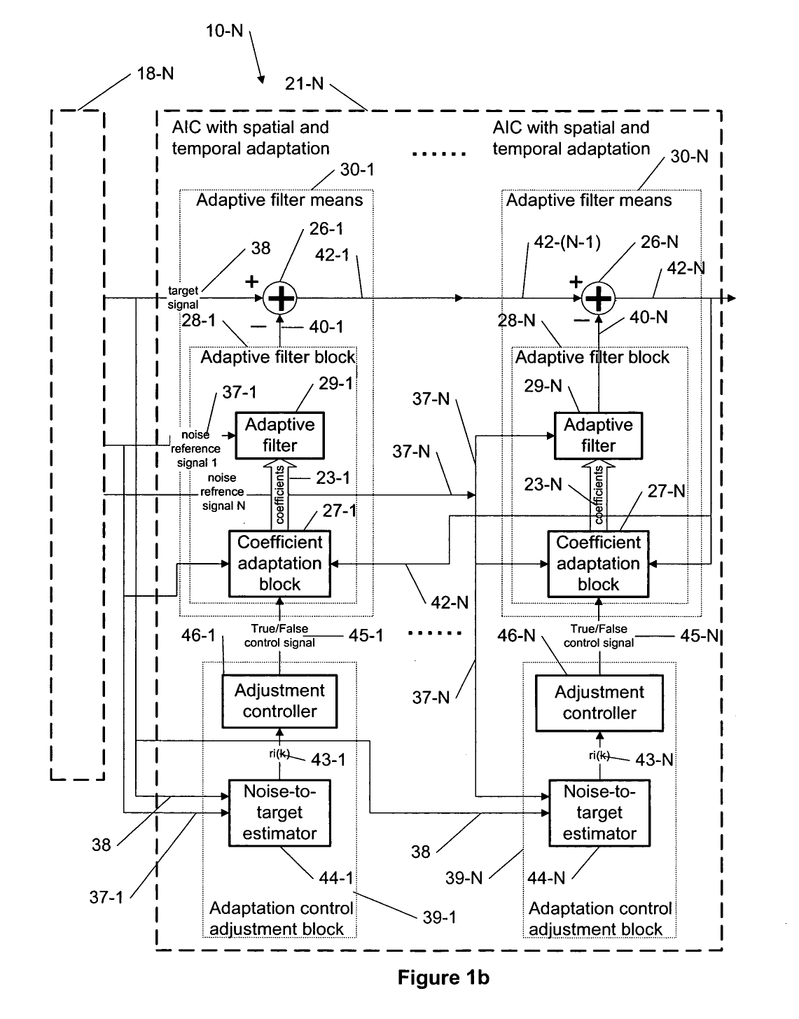

FIGS. 1a and 1b together demonstrate a block diagram representing one example among others of a generalized sidelobe canceling system 10-N with N noise reference signals 37-1, 37-2, . . . , 37-N using dynamic adjustment of adaptation control of an adaptive interference canceller 21-N based on spatially weighted beamforming pre-processing.

FIG. 1a shows components of the generalized sidelobe canceling system 10-N including a polynomial beamformer 18-N, which generates N noise reference signals 37-1, 37-2, . . . , 37-N supporting operation of the adaptive interference canceller 21-N shown in FIG. 1b, according to the present invention.

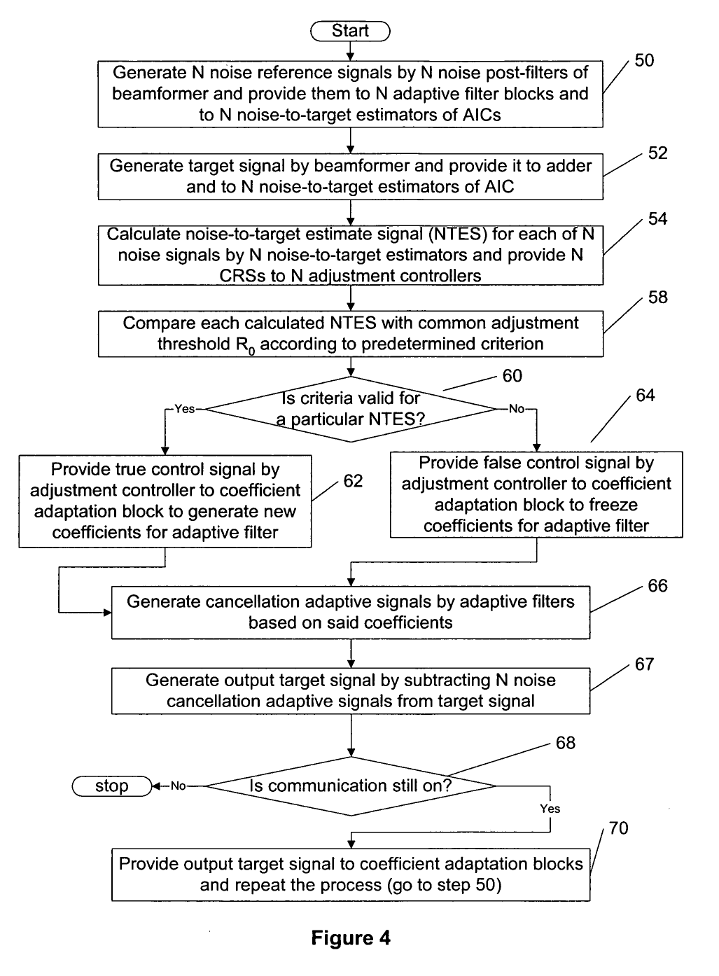

An acoustic signal 11 (see FIG. 1a) is received by a microphone array 12 with M microphones for generating M corresponding microphone (electro-acoustical) signals 30, wherein M is a finite integer of at least a value of two. Typically, the microphones in the microphone array 12 are arranged in a single array substantially along a horizontal line. However, the microphone can be arranged along a different direction, or in a 2D or 3D array. The M corresponding microphone signals 30 can be converted to digital signals 32 using an A/D converter 14 and each of said M digital microphone signals 32 is provided to each of T+1 pre-filters 20 of the polynomial beamformer 18-N, wherein T is a finite integer of at least a value of one. Operation of the polynomial beamformer 18-N and its components including T+1 pre-filters 20, a target post-filter 24, N noise post-filters 25-1, 25-2, . . . , 25-N, and a beam shape control block 22 are described in detail in European Patent No. 1184676 “A method and a Device for Parametric Steering of a Microphone Array Beamformer” by M. Kajala and M. Hämäläinen (corresponding PCT Patent Application publication WO 02/18969). Thus, the performance of the polynomial beamformer 18-N and its components are incorporated here by reference (see FIG. 4 and operation of the beamformer 30-11 of the above reference). The T+1 pre-filters 20 generate T+1 intermediate signals 34 in response to said M digital microphone signals 32 and provide T+1 intermediate signals 34 to the target post-filter 24 and to each of the N noise post-filters 25-1, 25-2, . . . , 25-N, said T+1 pre-filters 20, said target post-filter 24 and said noise post-filters 25-1, 25-2, . . . , 25-N are components of the beamformer 18-N, and N is a finite integer of at least a value of one. Said T+1 intermediate signals 34 are also provided to a speaker and noise tracking block 16 by the T+1 pre-filters 20.

The T+1 intermediate signals 34 still contain the spatial information of the M microphone signals 30 but in a different format. These T+1 intermediate signals 34 need to be further processed by the post-filters (24, 25-1, 25-2, . . . , 25-N) in order to achieve the signals that properly represent the look (target) directions specified by direction control signals (36, 36-1, 36-2, . . . 36-N) that are generated by a beam shape control block 22 as discussed below.

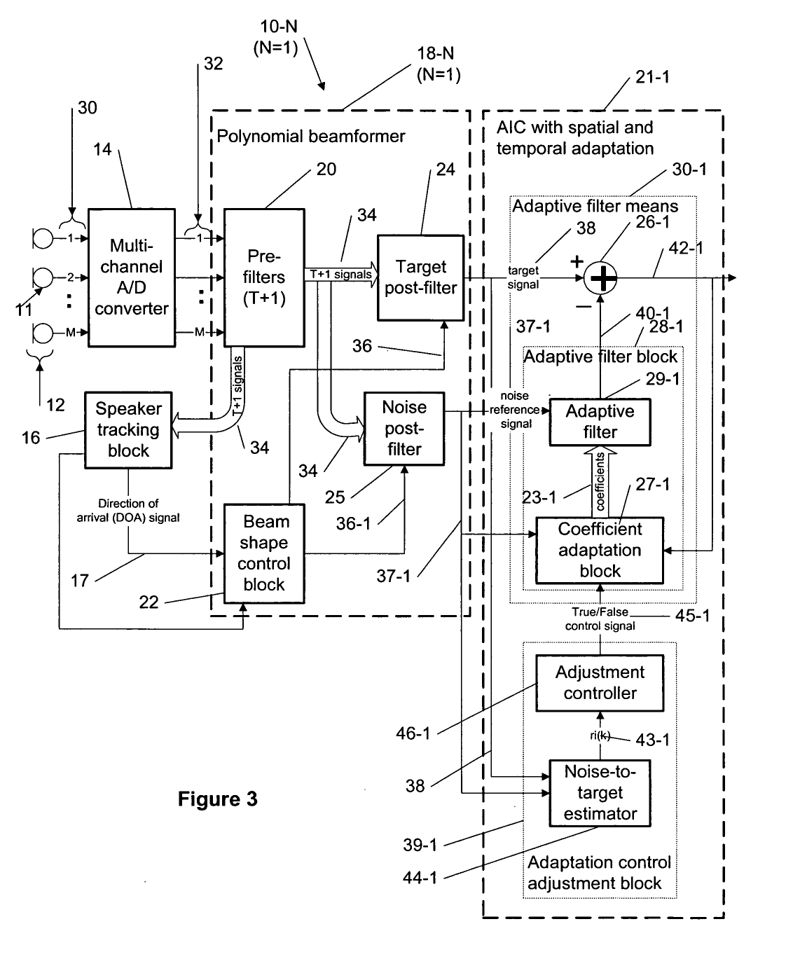

The performance of the speaker and noise tracking block 16 are described in U.S. Pat. No. 6,449,593 “Method and System for Tracking Human Speakers” by P. Valve are incorporated here by reference (see FIG. 3 of the above reference). The speaker and noise tracking block 16 is primarily used to select a favorable beam direction to track the speaker who speaks and the block 16 generates a direction of arrival (DOA) signal 17, and optionally (as discussed below) a noise direction signal 17a providing said direction of arrival signal 17 and optionally said noise direction signal 17a to the beam shape control block 22 (its performance is incorporated here by reference as stated above) of the polynomial beamformer 18-N. The speaker and noise tracking block 16 are able to trace a desired target signal direction and optionally noise signal directions as discussed below. The beam shape control block 22 generates a target control signal 35 and N noise control signals 36-1, 36-2, . . . 36-N and provides said control signals 35, 36-1, 36-2, . . . 36-N to the target post-filter 24 and to the N noise post-filters 25-1, 25-2, . . . , 25-N, respectively.

There are other methods, which can be used for generating the direction of arrival signal 17 as well as the noise direction signals 17a. It is noted that, according to the present invention, the location of the target signal source (and/or noise sources), i.e. forming the control signal 35 (and/or 36-1, 36-2, . . . 36-N), can be determined by checking the visual information obtained from a camera (if there is one attached to the system 10-N) or by any other means that can give the required information instead of using the speaker and noise tracking block 16.

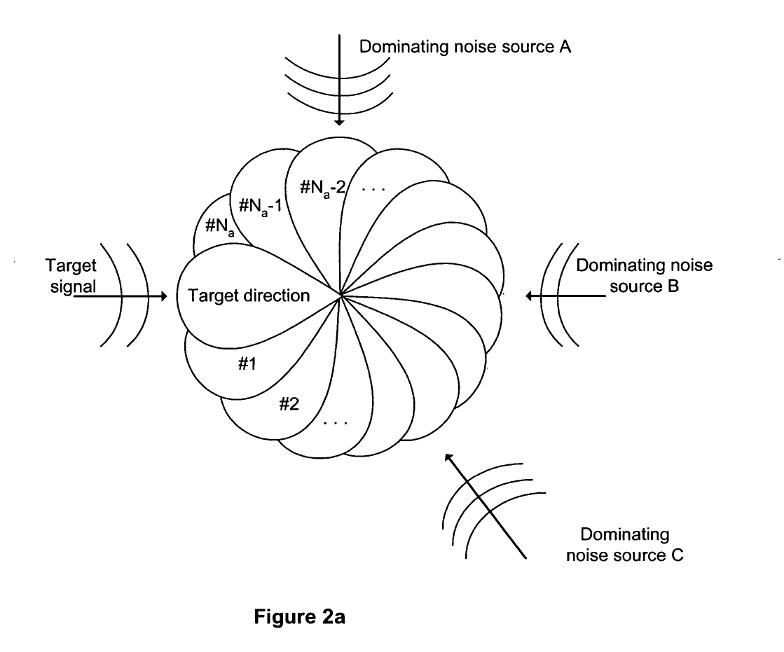

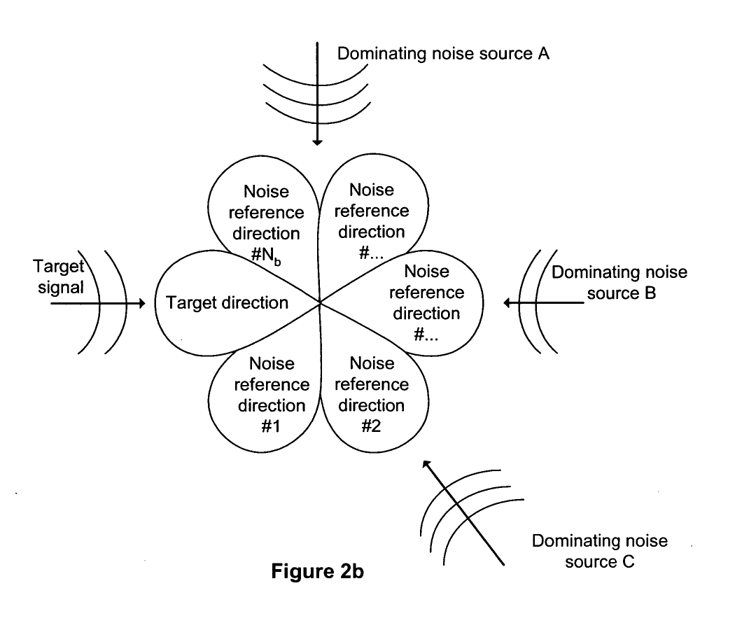

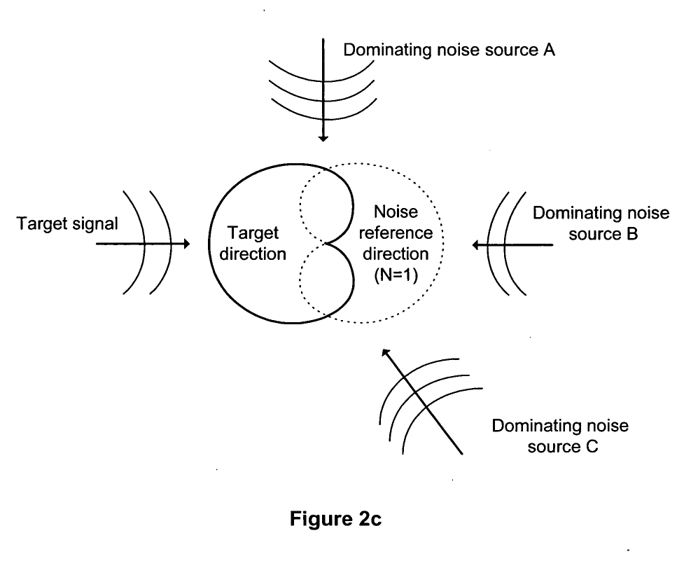

Noise reference direction estimation (the noise direction signals 17a) by the block 16 may not be necessarily needed, and therefore is optional according to the present invention, because the noise reference directions can be adjusted by generating N noise control signals 36-1, 36-2, . . . 36-N in accordance with the target signal direction (direction of arrival signal 17 or equivalent) in the beam shape control block 22 to cover the entire space of interest but steered away from a target direction as illustrated in FIG. 2 and discussed below. However, in some cases, e.g. if there exists external information about a strong interference direction, the use of the speaker and noise tracking block 16 (possibly receiving information from an external source in this case, not shown in FIG. 1a) for generating the noise direction signals 17a can improve the noise cancellation performance of the adaptive interference canceller (AIC) 21-N shown in FIG. 1b and discussed below. Also, generating signals 17a can be helpful if the entire space is not covered by the noise reference beams as shown in FIG. 2, wherein a dominating noise source A happens to fall in between the two consequent noise reference beams in a uniformly distributed beam space.

Further processing proceeds as follows. The target post-filter 24 generates a target signal 38 using the target control signal 35 and provides said target signal 38 to an adder 26-1 of the adaptive interference canceller 21-N. Each of the N noise post-filters 25-1, 25-2, . . . , 25-N generates a corresponding one of the N noise reference signals 37-1, 37-2, . . . , 37-N, respectively, by the N noise post-filters 25-1, 25-2, . . . , 25-N and provides the corresponding one of said N noise reference signals 37-1, 37-2, . . . , 37-N to a corresponding one of N adaptive filter blocks 28-1, 28-1, . . . , 28-N of the AIC 21-N shown in FIG. 1b, respectively, said N noise reference signals 37-1, 37-2, . . . , 37-N are steered away from the direction of a desired signal and, thus, the desired signal content is suppressed (blocked) in said N noise reference signals 37-1, 37-2, . . . , 37-N.

As stated above, the information about the target signal direction (or target DOA) is determined by the block 16 or other means described above. However, it is important that the noise reference directions of the N noise post-filters (25-1, 25-2, . . . , 25-N) are steered away from that direction. One possibility for achieving said steering is to steer the noise reference directions uniformly (or with some predetermined fixed distribution), preferably opposite to the look (target) direction as shown in FIG. 2, according to the present invention. The other possibility is to use the speaker and noise tracking block 16 (or alternatively an additional noise tracking block, not shown in FIG. 1) to generate the noise control signals 17a and subsequently the N noise control signals 36-1, 36-2, . . . 36-N that are used for generating the N noise reference signals 37-1, 37-2, . . . , 37-N.

FIG. 1b shows a block diagram of the adaptive interference canceller (AIC) 21-N of the generalized sidelobe canceling system 10-N performing dynamic adjustment of adaptation control. The AIC 21-N contains N sequentially arranged blocks as shown in the example of FIG. 1b. Each of said blocks comprises a corresponding one of the N adaptive filter means 30-1, 30-2, . . . , 30-N and a corresponding one of N adaptation control adjustment blocks 39-1, 39-2, . . . , 39-N, respectively. Each of the N adaptive filter means 30-1, 30-2, . . . , 30-N comprises a corresponding one of N adders 26-1, 26-2, . . . , 26-N and a corresponding one of the N adaptive filter blocks 28-1, 28-2, . . . , 28-N, respectively.

Each of the N adaptive filter blocks 28-1, 28-2, . . . , 28-N contains a corresponding one of N adaptive filters 29-1, 29-2, . . . , 29-N (e.g., FIR, finite-duration impulse response) and a corresponding one of N coefficient adaptation blocks 27-1, 27-2, . . . , 27-N, respectively. Each of the N noise reference signals 37-1, 37-2, . . . , 37-N is provided to the corresponding one of the N adaptive filters 29-1, 29-2, . . . , 29-N and to the corresponding one of the N coefficient adaptation blocks 27-1, 27-2, . . . , 27-N, respectively. Each of the N coefficient adaptation blocks 27-1, 27-2, . . . , 27-N is also provided with the output target signal 42-N and generates a corresponding one of N corresponding coefficient signals 23-1, 23-2, . . . , 23-N and provides one of said coefficient signals to the corresponding one of the N corresponding adaptive filters 29-1, 29-2, . . . , 29-N. Then each of the N adaptive filters 29-1, 29-2, . . . , 29-N generates a corresponding one of the N noise cancellation adaptive signals 40-1, 40-2, . . . , 40-N and provides the corresponding one of the said N noise cancellation adaptive signals 40-1, 40-2, . . . , 40-N to the corresponding one of the N adders 26-1, 26-2, . . . , 26-N, respectively. Each of the sequentially arranged N two-input adders 26-1, 26-2, . . . , 26-N subtracts the corresponding one of the noise cancellation adaptive signals 40-1, 40-2, . . . , 40-N from the target signal 38 (for the adder 26-1) or from one of corresponding N−1 intermediate output target signals 42-1, 42-2, . . . , 42-(N−1), respectively, to finally generate the output target signal 42-N. As an alternative to the preferred embodiment of FIG. 1b, the subtraction from the target signal 38 of the N corresponding noise cancellation adaptive signals 40-1, 40-2, . . . , 40-N for generating the output target signal 42-N can be alternatively performed just by one multi-input adder instead of the N two-input adders 26-1, 26-2, . . . , 26-N.

The described performance of the AIC 21-N without activating the N adaptation control adjustment blocks 39-1, 39-2, . . . , 39-N, rely only on spatial blocking of the target signal 38 from the N noise reference signals 37-1, 37-2, . . . , 37-N. The key innovation, according to the present invention, is achieved by introducing temporal adjustment of adaptation control of the AIC 21-N facilitated by the adaptation control adjustment blocks 39-1, 39-2, . . . , 39-N such that coefficient adaptation is inhibited while there is a clear presence (leakage) of desired signal components in the noise reference signals 37-1, 37-2, . . . , 37-N based on spatially weighted beamforming pre-processing, as described herein.

It is noted that typically there is always some leakage of desired signal content into the noise reference signals 37-1, 37-2, . . . , 37-N, but for a minor desired signal leakage it does not need to restrict adjustment of adaptive filter coefficients as long as the leaked desired signal component is masked by relatively strong interference, that is a noise-to-target estimate of the noise reference signal level to the target direction signal level is higher than the threshold level designed for the system. Said threshold level depends on the design of the beamformer front-end, e.g. beam shape in different directions, and a criterion of how much desired signal deterioration can be accepted at the output target signal 42 in order to maximize the signal-to-noise-ratio. It is also noted that coefficient adaptation control can only benefit from having a prior knowledge about the desired signal source characteristics in comparison to the noise (interference) source characteristics. Each of the N adaptation control adjustment blocks 39-1, 39-2, . . . , 39-N contains a corresponding one of N noise-to-target estimators 44-1, 44-2, . . . , 44-N and a corresponding one of N adjustment controllers 46-1, 46-2, . . . , 46-N. Also, each of the adaptation control adjustment blocks 39-1, 39-2, . . . , 39-N is provided with the target signal 38 and with the corresponding one of the N noise reference signals 37-1, 37-2, . . . , 37-N, respectively. Then, according to the present invention, the N noise-to-target estimators 44-1, 44-2, . . . , 44-N calculate corresponding N noise-to-target estimate signals 43-1, 43-2, . . . , 43-N.

The control ratio level rᵢ(k), corresponding to the N noise-to-target estimate signals 43-1, 43-2, . . . , 43-N with i=1, 2 . . . , N for the example of FIG. 1, is the ratio of the power of a noise reference, nᵢ(k) corresponding to the N noise reference signals 37-1, 37-2, . . . , 37-N, to the power of a desired signal, b(k), corresponding to the target signal 38, equals to

$${r}_{i} ( k ) = \frac{\overline{{n}_{i}^{2} ( k )}}{{b}^{2} ( k )} , i = 1 , … , N , \qquad ( 1 )$$

wherein k is a time index and i is a noise reference index.

The short-time power estimates of nᵢ(k) and b(k) can be calculated, e.g. by using the following iteration formula:
\overline{nᵢ²(k)}=(1−γ)nᵢ²(k)+γ\overline{nᵢ²(k−1)}and (2)
\overline{b²(k)}=(1−γ)b²(k)+γ\overline{b²(k−1)}, 0<<γ<1 (3)
wherein 0<<γ<1 is a smoothing coefficient, \overline{nᵢ²(0)}=0 and \overline{b²(0)}=0 are the initial conditions.

Then each of the N noise-to-target estimate signals 43-1, 43-2, . . . , 43-N is provided to a corresponding one of the N adjustment controllers 46-1, 46-2, . . . , 46-N which compares said corresponding one of the N noise-to-target estimate signals 43-1, 43-2, . . . , 43-N with a common adjustment threshold R₀ (e.g., R₀=1), according to a predetermined criterion. For example, if rᵢ(k)≧R₀ for any of the N noise-to-target estimate signals 43-1, 43-2, . . . , 43-N, e.g. when \overline{nᵢ²(k)}≧\overline{b²(k)} (R₀=1), the adaptation of the corresponding adaptive filter means out of N adaptive filter means 30-1, 30-2, . . . , 30-N, respectively, is allowed and the corresponding one of the N adjustment controllers 46-1, 46-2, . . . , 46-N provides a corresponding one of the true control signals (or in general case one of the corresponding adjustment signals) 45-1, 45-2, . . . , 45-N to a corresponding one of the N coefficient adaptation blocks 27-1, 27-2, . . . , 27-N and a spatial adaptation proceeds as described above. If, on the other hand, rᵢ(k)<R₀ for any of the N noise-to-target estimate signals 43-1, 43-2, . . ., 43-N, e.g. when \overline{nᵢ²(k)}<\overline{b²(k)} (R₀=1), the adaptation of the corresponding one of the N adaptive filter means 30-1, 30-2, . . . , 30-N, respectively, is not allowed and the corresponding one of the N adjustment controllers 46-1, 46-2, . . . , 46-N sends the corresponding one of the false control signals (or again in general case one of the corresponding adjustment signals) 45-1, 45-2, . . . , 45-N to freeze the coefficients to the corresponding one of the coefficient adaptation blocks 27-1, 27-2, . . . , 27-N, respectively, and the adaptation procedure is temporally frozen, i.e. filtering is still performed, but now with the fixed filter coefficients corresponding to the situation when the criterion of rᵢ(k)≧R₀ was last met. Thus, in addition to spatial adaptation based on spatially weighted beamforming pre-processing, temporal adjustment (or temporal blocking for the example of FIG. 1) of the adaptation control is facilitated by incorporating the N adaptation control adjustment blocks 39-1, 39-2, . . . , 39-N in the AIC 21-N.

Note that if we choose R₀=0, the AIC filter coefficients would be updated continuously (i.e. all the time) and, in case there is only a desired target signal present (no background noise), the desired speech signal would be deteriorated due to the fact that acoustical reflections of the desired signal tend to appear in the N noise reference signals 37-1, 37-2, . . . , 37-N, each which is an input to a corresponding one of the N adaptive filter blocks 28-1, 28-2, . . . , 28-N. On the other hand, if we choose a very high threshold for the common adjustment threshold, e.g. R₀=10, it can happen that all N noise-to-target estimate signals 43-1, 43-2, . . . , 43-N never exceed the given common adjustment threshold R₀, then, the AIC 21-N does not perform coefficient adaptation (those coefficients are frozen as described above) and there is no effective noise cancellation in the adders 26-1, 26-2, . . . , 26-N. Hence, a practical range for R₀ can be in the range 0.5≦R₀≦2.0, but the value of R₀ is not limited to that.

FIG. 1 represents just one simple example for implementing the present invention. There are many variations. Some of these variations are disclosed below.

In some applications it can be beneficial to set individual adjustment thresholds R₁, R₂, . . . , R_{N} for each of the N adjustment controllers 46-1, 46-2, . . . , 46-N, respectively. This can correspond to the case of different beam widths in different directions. For example, it is well-known that linear arrays of omni-directional microphones produce efficient cone-like beams in the end-fire direction but, on the other hand, the broadside response is a very excessive donut-like pattern. That naturally causes different beam output powers depending on where the actual signal source is located with respect to the orientation of the array and, thus, the target direction power to noise direction adjustment thresholds should be defined separately for each noise reference direction in relation to the microphone array (i.e. taking into account the beam shape in that direction) and in relation to the target signal direction (target beam shape changes as well when the target is moving and the target beam is steered in different directions).

In another scenario, depending on the application, the common adjustment threshold R₀ or alternatively, adjustment thresholds R₁, R₂, . . . , R_{N} described above can be time varying depending, e.g. on changes in the target and noise direction beam shapes or adopting to changes in the acoustical environment. It can be the case that in a quiet environment it is desirable to preserve the desired speech signal (target) as clean as possible and thus the value of the common adjustment threshold R₀ or the adjustment thresholds R₁, R₂, . . . , R_{N} will be set high enough to prevent adaptive filters from adapting the target signal 38 in order to produce the output target signal 42. On the other hand, in extreme ambient noise conditions, it can be better to try to catch at least some of the desired speech from the heavy background noise to make the speech signal as intelligible as possible and, thus, the value of the common adjustment threshold R₀ or adjustment thresholds R₁, R₂, . . . , R_{N} may need to be set very small (some deterioration of the target speech signal would be acceptable in order to reduce the noise as much as possible).

Yet, another possible variation of the basic example of FIG. 1 can involve system performance improvement not only by a restricted ON/OFF type of threshold but by using a more generalized adaptation control, e.g. a smoothly changing adaptation rate control using, for example, an adaptive filter coefficient adaptation step size. Thus, for example, the adaptation control can be smoothly accelerating or inhibiting filter coefficient adaptation by one of the N coefficient adaptation blocks 27-1, 27-2, . . . , 27-N when the corresponding noise-to-target estimate signal of the N noise-to-target estimate signals 43-1, 43-2, . . . , 43-N is within a certain boundary (generally it can be multiple boundaries corresponding to different levels of adjusting adaptation control for each of the N noise-to-target estimate signals 43-1, 43-2, . . . , 43-N) determined for each of the N noise directions based on a predetermined criterion. Yet in another possible scenario said multiple boundaries can be time-varying as well based on a further predetermined criterion. Adaptation rate control of conventional adaptive filters is well known in the art, see e.g. Haykin S., “Adaptive Filter Theory”, Prentice-Hall, 4th Edition, 2002, Section 6.3, pp. 327-331.

Yet still another possible variation of the present invention is implementation of the present invention (for example shown in FIG. 1) in a frequency domain or in a time domain or in both domains.

FIGS. 2a, 2b and 2c illustrate different examples of distribution of a target direction and noise reference directions, according to the present invention.

FIG. 2a gives an example of a uniform spatial distribution in 2D space of Nₐ noise reference acoustical directions that cover the entire acoustical space around the microphone array 12. FIG. 2a shows a target acoustical signal, three dominating noise sources (A, B and C), target direction receiving sensitivity profile and N fixed noise reference direction sensitivity profiles (in relation to the detected target direction). Note that, for simplicity, the drawing does not show the sidelobes of the individual sensitivity patterns.

FIG. 2b is similar to 2a, but with a reduced coverage of N_{b}(N_{b}<Nₐ) noise reference acoustical directions, wherein a spatial null appears in the direction of the noise source A. So, the noise source directions are not steered independently and it can be seen that, e.g. one noise source (the acoustical signal from the source A) falls between two noise reference beams and is not perhaps quite optimally picked-up.

FIG. 2c is an illustration of extremely reduced coverage of the noise reference acoustical directions having only one target signal direction and a single noise reference direction (N=1) and using a very simple cardioid sensitivity pattern for the sound pick-up, according to the present invention. It can be seen that in this case the single noise reference signal does not spatially separate the noise sources A, B and C, but the resulting noise reference signal is still blocking the target signal, which is the major issue in the present invention.

One important consideration regarding the noise reference beams is the ability to block out the target signal, which is important to guarantee proper operation of the AIC block 21-N. Also, the set of N noise reference beams still approximately covers the entire space around the microphone array 12 in order to receive one or more actual noise source signals A, B, etc. As described above, if there exists external information about a strong interference direction (e.g., dominating noise sources A, B and/or C of FIGS. 2a, 2b and 2c), the use of the speaker and noise tracking block 16 for generating the noise direction signals 17a can improve the noise cancellation performance of the adaptive interference canceller block 21-N.

FIG. 3 is a block diagram representing one example among others of generalized sidelobe canceling with only one noise reference signal, using temporal (dynamic) adjustment of adaptation control of the adaptive interference canceller 21-N based on spatially weighted beamforming pre-processing, according to the present invention. Instead of the N noise post-filters 25-1, 25-2, . . . , 25-N, N adaptive filter blocks 28-1, 28-1, . . . , 28-N, N adaptation control adjustment blocks 39-1, 39-2, . . . , 39-N and N adders 26-1, 26-2, . . . , 26-N, there is only one noise post-filter 25-1, one adaptive filter block 28-1, one adaptation control adjustment block 39-1 and one adder 26-1, respectively, which simplifies the system performance.

FIG. 4 shows a flow chart of generalized sidelobe canceling using temporal (dynamic) adjustment of adaptation control of the adaptive interference canceller 21-N based on spatially weighted beamforming pre-processing presented in FIGS. 1a and 1b, according to the present invention. The flow chart of FIG. 4 only represents one possible scenario among others. In a method according to the present invention, in a first step 50, the N noise reference signals 37-1, 37-2, . . . , 37-N are generated by the beamformer 18-N and provided to the corresponding N adaptive filter blocks 28-1, 28-1, . . . , 28-N (including both the N adaptive filters 29-1, 29-2, . . . , 29-N and N coefficient adaptation blocks 27-1, 27-2, . . . , 27-N, respectively) and to the corresponding N noise-to-target estimators 44-1, 44-2, . . . , 44-N. In a next step 52, the target signal 38 is generated by the beamformer 18-N and provided to the adder 26-1 and to each of the N noise-to-target estimators 44-1, 44-2, . . . , 44-N of the AIC 21-N. In a next step 54, the N noise-to-target estimate signals 43-1, 43-2, . . . , 43-N are generated by the corresponding N noise-to-target estimators 44-1, 44-2, . . . , 44-N and each of said noise-to-target estimate signals 43-1, 43-2, . . . , 43-N is provided to the corresponding one of the N adjustment controllers 46-1, 46-2, . . . , 46-N. In a next step 58, each of the N noise-to-target estimate signals 43-1, 43-2, . . . , 43-N is compared with the common adjustment threshold R₀ by the corresponding one of the N adjustment controllers 46-1, 46-2, . . . , 46-N according to a predetermined criterion.

In a next step 60, it is ascertained whether said criterion is met for each of the N adjustment controllers 46-1, 46-2, . . . , 46-N. If that is the case for any of the adjustment controllers 46-1, 46-2, . . . , 46-N, in a next step 62, said adjustment controller provides the true control signal (or in general case the adjustment signal) which is one of the N signals 45-1, 45-2, . . . , 45-N to the corresponding one of the N coefficient adaptation blocks 27-1, 27-2, . . . , 27-N, respectively, and adaptation proceeds normally. If, however, the criterion is not met for any of the adjustment controllers 46-1, 46-2, . . . , 46-N, in a next step 64, said adjustment controller provides the false control signal (or in general case again the adjustment signal) which is one of the N signals 45-1, 45-2, . . . , 45-N) to a corresponding one of the N coefficient adaptation blocks 27-1, 27-2, . . . , 27-N, respectively, which freezes the adaptation coefficients of that block and subsequently the adaptation process. In a next step 66, the N cancellation adaptive signals 40-1, 40-2, . . . , 40-N are generated by the corresponding N adaptive filters 29-1, 29-2, . . . , 29-N based on the corresponding coefficient signals 23-1, 23-2, . . . , 23-N, provided to each of the N adaptive filters 29-1, 29-2, . . . , 29-N by the corresponding N coefficient adaptation blocks 27-1, 27-2, . . . , 27-N, respectively.

In a next step 67, the output target signal 42-N is generated by subtracting each of the N noise cancellation adaptive signals 40-1, 40-2, . . . , 40-N from the target signal 38 by the corresponding consecutively arranged N adders 26-1, 26-2, . . . , 26-N. In a next step 68, it is ascertained whether the communication is still on. If that is not the case, the process stops. If, however, the communication is still on, in a next step 70, the output target signal 42-N is provided as a feedback to each of the coefficient adaptation blocks 27-1, 27-1, . . . , 27-N of the respective adaptive filter blocks 28-1, 28-1, . . . , 28-N and the process goes back to step 50.

1. A method for dynamic adjustment of adaptation control of an adaptive interference canceller (21-N) based on spatially weighted beamforming pre-processing, comprising the steps of:
generating (50, 52) a target signal (38) and N noise reference signals (37-1, 37-2, . . . , 37-N) by a beamformer (18-N) and providing said target signal (38) and said N noise reference signals (37-1, 37-2, . . . , 37-N) to the adaptive interference canceller (21-N), wherein N is a finite integer of at least a value of one;
calculating (54) by the adaptive interference canceller (21-N) N noise-to-target estimate signals (43-1, 43-2, . . . , 43-N) and comparing (58), according to a predetermined criterion, each of the N noise-to-target estimate signals (43-1, 43-2, . . . , 43-N) with a corresponding one of N adjustment thresholds (R_{1, R}₂, . . . , R_{N}), respectively, and optionally with at least one further adjustment threshold, wherein said at least one further adjustment threshold is selected individually for each of the N noise-to-target estimate signals (43-1, 43-2, . . . , 43-N);
providing (62, 64), based on said predetermined criterion, each of N adjustment signals (45-1, 45-2, . . . , 45-N) to a corresponding one of N adaptive filter blocks (28-1, 28-2, . . . , 28-N) of the adaptive interference canceller (21-N), respectively;
generating (66) each of N noise cancellation adaptive signals (40-1, 40-2, . . . , 40-N) by the corresponding one of the N adaptive filter blocks (28-1, 28-2, . . . , 28-N) based on a corresponding one of the N adjustment signals (45-1, 45-2, . . . , 45-N); and
generating (70) an output target signal (42-N) by subtracting all N noise cancellation adaptive signals (40-1, 40-2, . . . , 40-N) from the target signal (38).

2. The method of claim 1, wherein the beamformer (18-N) is a polynomial beamformer.

3. The method of claim 1, wherein the target signal (38) is generated by a target post-filter (24) of the beamformer (18-N) in response to each of T+1 intermediate signals (34) and to a target control signal (35), and each of the N noise reference signals (37-1, 37-2, . . . , 37-N) is generated by each of the N noise post-filters (25-1, 25-1, . . . , 25-N) of the beamformer (18-N) in response to the T+1 intermediate signals (34) and to a corresponding one of the N noise control signals (36-1, 36-2, . . . 36-N) provided to a corresponding one of the N noise post-filters (25-1, 25-1, . . . , 25-N), respectively, said T+1 intermediate signals are generated by T+1 pre-filters (20) of the beamformer (18-N), each of said T+1 pre-filters (20) is responsive to M microphone signals (30) or to M digital microphone signals (32), and said target control signal (35) and said noise control signals (36-1, 36-2, . . . 36-N) are generated by a beam shape control block (22) of the beamformer (18-N), wherein M is a finite integer of at least a value of two and T is a finite integer of at least a value of one.

4. The method of claim 3, wherein the M microphone signals (30) are generated by a microphone array (12) containing M microphones, responsive to an acoustic signal (11).

5. The method of claim 3, wherein the M digital microphone signals (32) are generated by an A/D converter (14) from the M microphone signals (30) provided by the microphone array (12).

6. The method of claim 1, wherein said target signal (38) and a corresponding one of said N noise reference signals (37-1, 37-2, . . . , 37-N) are provided to each of N noise-to-target estimators (44-1, 44-2, . . . , 44-N) of the adaptive interference canceller (21-N), respectively, and each of N noise-to-target estimate signals (43-1, 43-2, . . . , 43-N) is calculated by a corresponding one of the N noise-to-target estimators (44-1, 44-2, . . . , 44-N) as a ratio of the corresponding one of the N noise reference signals (37-1, 37-2, . . . , 37-N), respectively, and the target signal (38).

7. The method of claim 1, wherein each of the N adjustment signals (45-1, 45-2, . . . , 45-N) is a true/false control signal and each of the N noise-to-target estimate signals (43-1, 43-2, . . . , 43-N) is only compared, according to a predetermined criterion, with the corresponding one of the N adjustment thresholds (R₁, R₂, . . . , R_{N}), respectively.

8. The method of claim 7, wherein all of the N adjustment thresholds (R₁, R₂, . . . , R_{N}) are equal to each other and to a common adjustment threshold (R₀).

9. The method of claim 8, wherein the common adjustment threshold (R₀) is in the range 0.5≦R₀≦2.0.

10. The method of claim 7, wherein each of the N true/false control signals (45-1, 45-2, . . . , 45-N) is determined by comparing a corresponding one of the N noise-to-target estimate signals (43-1, 43-2, . . . , 43-N) with the corresponding one of the N adjustment thresholds (R₁, R₂, . . . , R_{N}), respectively, such that if any of said noise-to-target estimate signals (43-1, 43-2, . . . , 43-N) is larger than the corresponding one of the N adjustment thresholds (R₁, R₂, . . . , R_{N}), the corresponding true control signal is provided to a corresponding one of the N adaptive filter blocks (28-1, 28-2, . . . , 28-N), respectively, but if any of said N noise-to-target estimate signals (43-1, 43-2, . . . , 43-N) is smaller than the corresponding one of the N corresponding adjustment thresholds (R₁, R₂, . . . , R_{N}), then the false control signal is provided to the corresponding one of the N adaptive filter blocks (28-1, 28-2, . . . , 28-N), respectively.

11. The method of claim 10, wherein each of the N true/false control signals (45-1, 45-2, . . . , 45-N) is used for adjusting an adaptation rate of the corresponding one of the N adaptive filter blocks (28-1, 28-2, . . . , 28-N) of the adaptive interference canceller (21-N), respectively.

12. The method of claim 10, wherein the N true/false control signals (45-1, 45-2, . . . , 45-N) are provided to the N adaptive filter blocks (28-1, 28-2, . . . , 28-N) for enabling or disabling the adaptation control of adaptation coefficients to allow generating new adaptation coefficients in case of the true control signals (45-1, 45-2, . . . , 45-N) or freezing said adaptation coefficients in case of the false control signal (45-1, 45-2, . . . , 45-N) by each of the N adaptive filter blocks (28-1, 28-2, . . . , 28-N).

13. The method of claim 1, wherein each of N coefficient adaptation blocks (27-1, 27-2, . . . , 27-N) of the corresponding one of the N adaptive filter blocks (28-1, 28-2, . . . , 28-N) provides a corresponding one of N coefficient signals (23-1, 23-2, . . . , 23-N) to a corresponding one of N adaptive filters (29-1, 29-2, . . . , 29-N), respectively, in response to a corresponding one of the N noise reference signals (37-1, 37-2, . . . , 37-N) and to the output target signal (42-N), and wherein each of the N adaptive filters (29-1, 29-2, . . . , 29-N) provides a corresponding one of the N noise cancellation adaptive signals (40-1, 40-2, . . . , 40-N) to a corresponding one of N adders (26-1, 26-2, . . . , 26-N) in response to the corresponding one of the N noise reference signals (37-1, 37-2, . . . , 37-N) and to the corresponding one of the N coefficient signals (23-1, 23-2, . . . , 23-N), respectively.

14. The method of claim 13, wherein the N adaptive filters (29-1, 29-2, . . . , 29-N) are finite impulse response (FIR) filters.

15. The method of claim 1, wherein subtracting of the N noise cancellation adaptive signals (40-1, 40-2, . . . , 40-N) from the target signal (38) for generating the output target signal (42-N) is performed by N adders (26-1, 26-2, . . . , 26-N) sequentially by generating N−1 corresponding intermediate output target signals (42-1, 42-2, . . . , 42-(N−1)).

16. The method of claim 1, wherein subtracting the N noise cancellation adaptive signals (40-1, 40-2, . . . , 40-N) from the target signal (38) is performed by a combined adder.

17. The method of claim 1, wherein the output target signal (42-N) is provided to each of the N adaptive filter blocks (28-1, 28-1, . . . , 28-N) for continuing an adaptation process and for generating a further value of the output target signal (42-N).

18. The method of claim 1, wherein N=1

19. The method of claim 1, wherein at least one of the N adjustment thresholds (R₁, R₂, . . . , R_{N}) or of the at least one further adjustment threshold for any of the N noise-to-target estimate signals (43-1, 43-2, . . . , 43-N) is variable as a function of time according to a further predetermined criteria.

20. The method of claim 1, wherein all of said N adjustment thresholds (R₁, R₂, . . . , R_{N}) and the at least one further adjustment threshold for each of the N noise-to-target estimate signals (43-1, 43-2, . . . , 43-N) are variable as a function of time according to a further predetermined criteria.

21. The method of claim 1, wherein said at least one further adjustment threshold for each of the N noise-to-target estimate signals (43-1, 43-2, . . . , 43-N) is equal to a further common adjustment threshold.

22. The method of claim 1, wherein each of the N adjustment signals (45-1, 45-2, . . . , 45-N) is used for adjusting an adaptation rate of the corresponding one of the N adaptive filter blocks (28-1, 28-2, . . . , 28-N) of the adaptive interference canceller (21-N), respectively.

23. The method of claim 1, wherein an adaptive interference cancellation is performed in a frequency domain, or in a time domain or in both the frequency and the time domain.

24. A generalized sidelobe canceling system (10-N), comprising:
a beamformer (18-N), for providing a target signal (38) and N noise reference signals (37-1, 37-2, . . . , 37-N), wherein N is a finite integer of at least a value of one; and
an adaptive interference canceller (21-N), responsive to the target signal (38), to N noise reference signals (37-1, 37-2, . . . , 37-N) and to an output target signal (42-N), for adjusting adaptation control of the output target signal (42-N) based on calculating N noise-to-target estimate signals (43-1, 43-2, . . . , 43-N) and comparing, according to a predetermined criterion, each of said N noise-to-target estimate signals (43-1, 43-2, . . . , 43-N) with a corresponding one of N adjustment threshold (R₁, R₂, . . . , R_{N}), respectively, and optionally with at least one further adjustment threshold, wherein said at least one further adjustment threshold is selected individually for each of the noise-to-target estimate signals (43-1, 43-2, . . . , 43-N).

25. The generalized sidelobe canceling system (10-N) of claim 24, wherein the beamformer (18-N) is a polynomial beamformer.

26. The generalized sidelobe canceling system (10-N) of claim 24, wherein N=1.

27. The generalized sidelobe canceling system (10-N) of claim 24, further comprising:
a microphone array (12) containing M microphones, responsive to an acoustic signal (11), for providing M microphone signals (30), wherein M is a finite integer of at least a value of two;
an A/D converter (14), responsive to the M microphone signals (30), for providing M digital microphone signals (32); and
a speaker and noise tracking block (16), responsive to the T+1 intermediate signals (34), for providing a direction of arrival signal (17) and N noise direction signals (17a), wherein T is a finite integer of at least a value of one.

28. The generalized sidelobe canceling system (10-N) of claim 27, wherein the beamformer (18-N) is responsive to the M microphone signals (30) or to the M digital microphone signals (32) and optionally responsive to the direction of arrival signal (17) and to the N noise direction signals (17a), for providing T+1 intermediate signals (34), a target control signal (35) and N noise control signals (36-1, 36-2, . . . 36-N).

29. The generalized sidelobe canceling system (10-N) of claim 27, wherein the beamformer (18-N) comprises:
T+1 pre-filters (20), responsive to the M digital microphone signal (32), for providing the T+1 intermediate signals (34);
N target post-filters (24), responsive to the T+1 intermediate signals (34) and to the target control signal (35), for providing the target signal (38);
N noise post-filters (25-1, 25-1, . . . , 25N), each responsive to the T+1 intermediate signals (34) and to a corresponding one of the N noise control signals (36-1, 36-2, . . . 36-N), each for providing a corresponding one of the N noise reference signals (37-1, 37-2, . . . , 37-N); and
a beam shape control block (22), optionally responsive to the direction of arrival signal (17) and to the N noise direction signals (17a), for providing the target control signal (35) and the N noise control signals (36-1, 36-2, . . . 36-N).

30. The generalized sidelobe canceling system (10-N) of claim 24, wherein the adaptive interference canceller (21-N) comprises:
N adaptive filter blocks (28-1, 28-2, . . . , 28-N), each responsive to the output target signal (42-N), to a corresponding one of N adjustment signals (45-1, 45-2, . . . 45-N) and to a corresponding one of the N noise reference signals (37-1, 37-2, . . . , 37-N), respectively, each for providing one of N noise cancellation adaptive signals (40-1, 40-2, . . . , 40-N) by a corresponding one of the N adaptive filter blocks (28-1, 28-2, . . . , 28-N);
N consecutive adders (26-1, 26-2, . . . , 26-N), each responsive to the target signal (38) and to the corresponding one of the N noise cancellation adaptive signals (40-1, 40-2, . . . , 40-N), respectively, each for providing one of N−1 corresponding intermediate signals (42-1, 42-2, . . . , 42-(N−1)) or the output target signal (42-N) by a corresponding one of the N adders (26-1, 26-2, . . . , 26-N), respectively; and
N adaptation control adjustment blocks (39-1, 39-2, . . . , 39-N), each responsive to the target signal (38) and to the corresponding one of the N noise reference signals (37-1, 37-2, . . . , 37-N), respectively, each for providing one of the N corresponding adjustment signals (45-1, 45-2, . . . , 45-N) by a corresponding one of the N adaptation control adjustment blocks (39-1, 39-2, . . . , 39-N).

31. The generalized sidelobe canceling system (10-N) of claim 30, wherein each of the adaptive filter blocks (28-1, 28-2, . . . , 28-N) comprises:
an adaptive filter (29-1, 29-2, . . . , 29-N), responsive to the corresponding one of the N noise reference signals (37-1, 37-2, . . . , 37-N) and to a corresponding one of N coefficient signals (23-1, 23-2, . . . , 23-N), respectively, for providing one of the N noise cancellation adaptive signals (40-1, 40-2, . . . , 40-N) by a corresponding one of the N adaptive filters (29-1, 29-2, . . . , 29-N), respectively; and
a coefficient adaptation block (27-1, 27-2, . . . , 27-N), responsive to the corresponding one of the N noise reference signals (37-1, 37-2, . . . , 37-N), responsive to the output target signal (42-N), for providing one of the N coefficient signals (23-1, 23-2, . . . , 23-N) by a corresponding one of the N coefficient adaptation blocks (27-1, 27-2, . . . , 27-N), respectively.

32. The generalized sidelobe canceling system (10-N) of claim 30, wherein each of the N adaptation control adjustment blocks (39-1, 39-2, . . . , 39-N) comprises:
a noise-to-target estimator (44-1, 44-1, . . . , 44-N), responsive to the target signal (38) and to the corresponding one of the N noise reference signals (37-1, 37-2, . . . , 37-N), respectively, for providing a corresponding one of the N noise-to-target estimate signals (43-1, 43-2, . . . , 43-N), respectively; and
an adjustment controller (46-1, 46-2, . . . 46-N), responsive to the corresponding one of the N noise-to-target estimate signals (43-1, 43-2, . . . , 43-N), for providing a corresponding one of the N adjustment signals (45-1, 45-2, . . . , 45-N), respectively.

33. The generalized sidelobe canceling system (10-N) of claim 24, wherein all of the N adjustment thresholds (R₁, R₂, . . . , R_{N}) are equal to each other and to a common adjustment threshold (R₀).

34. The generalized sidelobe canceling system (10-N) of claim 33, wherein the common adjustment threshold (R₀) is in the range 0.5≦R₀≦2.0.

35. The generalized sidelobe canceling system (10-N) of claim 24, wherein said system (10-N) is implemented in a frequency domain, or in a time domain or in both the frequency and the time domain.

36. An adaptive interference canceller (21-N) for generating an output target signal (42-N) with dynamic adjustment of adaptation control, comprising:
N adaptive filter blocks (28-1, 28-2, . . . , 28-N), each responsive to the output target signal (42-N), to a corresponding one of N adjustment signals (45-1, 45-2, . . . , 45-N) and to a corresponding one of N noise reference signals (37-1, 37-2, . . . , 37-N), each for providing one of N noise cancellation adaptive signals (40-1, 40-2, . . . , 40-N) by a corresponding one of the N adaptive filter blocks (28-1, 28-2, . . . , 28-N); and
N adaptation control adjustment blocks (39-1, 39-2, . . . , 39-N), each responsive to the target signal (38) and to the corresponding one of the N noise reference signals (37-1, 37-2, . . . , 37-N), respectively, each for providing one of the N adjustment signals (45-1, 45-2, . . . , 45-N) by a corresponding one of the N adaptation control adjustment blocks (39-1, 39-2, . . . , 39-N).

37. The adaptive interference canceller (21-N) of claim 36, further comprising:
N consecutive adders (26-1, 26-2, . . . , 26-N), each responsive to the target signal (38) and to a corresponding one of the N noise cancellation adaptive signals (40-1, 40-2, . . . , 40-N), respectively, each for providing one of N−1 corresponding intermediate signals (42-1, 42-2, . . . , 42-(N−1)) or the output target signal (42-N) by a corresponding one of the N adders (26-1, 26-2, . . . , 26-N), respectively.

38. The adaptive interference canceller (21-N) of claim 36, wherein each of the N adaptive filter blocks (28-1, 28-2, . . . , 28-N) comprises:
an adaptive filter (29-1, 29-2, . . . , 29-N), responsive to a corresponding one of the N noise reference signals (37-1, 37-2, . . . , 37-N) and to a corresponding one of N coefficient signals (23-1, 23-2, . . . , 23-N), respectively, for providing one of the N noise cancellation adaptive signals (40-1, 40-2, . . . , 40-N) by a corresponding one of the N adaptive filters (29-1, 29-2, . . . , 29-N), respectively; and
a coefficient adaptation block (27-1, 27-2, . . . , 27-N), responsive to the corresponding one of the N noise reference signals (37-1, 37-2, . . . , 37-N) and to the output target signal (42-N), for providing one of the N coefficient signals (23-1, 23-2, . . . , 23-N) by a corresponding one of the coefficient adaptation blocks (27-1, 27-2, . . . , 27-N), respectively.

39. The adaptive interference canceller (21-N) of claim 36, wherein each of the N adaptation control adjustment blocks (39-1, 39-2, . . . , 39-N) comprises:
a noise-to-target estimator (44-1, 44-1, . . . , 44-N), responsive to the target signal (38) and to a corresponding one of the N noise reference signals (37-1, 37-2, . . . , 37-N), respectively, for providing a corresponding one of N noise-to-target estimate signals (43-1, 43-2, . . . , 43-N), respectively; and
an adjustment controller, responsive to the corresponding one of the N noise-to-target estimate signals (43-1, 43-2, . . . , 43-N), for providing a corresponding one of the N adjustment signals (45-1, 45-2, . . . , 45-N), respectively.
